(19) United States
(12) Patent Application Publication
(10) Pub. No.: US 2018/0243725 A1
(43) Pub. Date: Aug. 30, 2018

(54) SEPARATING AGENT FOR LIQUID CHROMATOGRAPHY, SEPARATION COLUMN, AND METHOD FOR SEPARATING AND PURIFYING BIOPOLYMER USING THEM

(71) Applicants: Shinwa Chemical Industries, Ltd., Kyoto-shi (JP); Kyoto University, Kyoto-shi (JP)

(72) Inventors: Hiroshi Kobayashi, Hirakata-shi (JP); Takuya Kubo, Kyoto-shi (JP); Koji Otsuka, Kyoto-shi (JP)

(21) Appl. No.: 15/891,223

(22) Filed: Feb. 7, 2018

(30) Foreign Application Priority Data
Feb. 8, 2017 (JP) 2017-021376

(51) Int. Cl. B01J 20/289; C07K 1/22; B01D 15/38

(52) U.S. Cl. CPC B01J 20/289; B01D 15/3823; C07K 1/22

(57) ABSTRACT

In order to provide a separating agent for liquid chromatography that is able to separate a protein using target characteristics as an index while retaining the original steric structure, the separating agent for liquid chromatography is equipped with a substrate, a recognition site including a compound that operates by recognizing characteristics of biopolymers such as proteins, and a spacer that bonds the recognition site to the substrate, wherein the spacer has an effective length to enable the recognition site to operate by reaching deep portions of the steric structure of a target biopolymer.

### TECHNICAL FIELD

The present invention relates to a separating agent and a separation column for liquid chromatography that are useful when separating and purifying biologically-derived polymers (hereinafter, referred to as biopolymers) such as proteins, as well as to a method for separating and purifying biopolymers that uses these.

### TECHNICAL BACKGROUND

In liquid chromatography, an adsorption agent into which a recognition site has been introduced is used as a separating agent, and a target compound is adsorbed using a separation column that has been filled with these, and separation is performed continuously at the same time as desorption is performed from differences in the solubility in an eluent.

For example, as is shown in Non-patent document 1, when separating small compounds at a tricyclic aromatic hydrocarbon level, the separation can be performed using a separating agent in which fullerene or the like has been directly bonded as the recognition site to the surface of a substrate.

Furthermore, a method such as that described in Non-patent document 2 in which graphene is fixed to the surface of a substrate may also be considered.

When biopolymers such as proteins and peptides are being separated using this type of conventional method, because the biopolymer has a coiled steric structure, as is shown in FIGS. 1(a) and (b), the area where the surface of the aforementioned separating agent for liquid chromatography, or the recognition site bonded to the surface of this separating agent, is able to operate is limited to superficial portions of this biopolymer having a steric structure.

Because of this, when characteristic portions created by post-translational modifications such as, for example, acylation, acetylation, alkylation, amidation, biotinylation, formylation, carboxylation, glutamylation, glycosylation, glycylation, hydroxylation, iodination, isoprenylation, lipoylation, phosphorylation, racemization, ubiquitination, and nitrosylation and the like that impart characteristic properties to proteins are present in deep portions of the biopolymer steric structure, then separation or purification of the biopolymers in which these characteristic portions are used as an index is difficult.

For reasons such as this, when, for example, a sugar chain is coupled by post-translational modification to a protein, in order to analyze the characteristics produced by the sugar chain and understand the operation thereof, conventionally, for example, as is shown in Patent document 1, processing that destroys the structure of the biopolymer is performed as preprocessing prior to the separation analysis performed using liquid chromatography. This processing may involve breaking down the proteins contained in a test sample into small fragments using proteolytic enzymes, and then cutting out the sugar chain using deglycosylation enzymes. Alternatively, the processing may involve directly cutting out the sugar chain by performing deglycosylation enzyme processing directly on the proteins, and then performing separation analysis thereon.

However, in the conventional method, because it is necessary to perform operations such as analyzing the proteins using proteolytic enzymes, and removing the proteolytic enzymes after the proteolysis, and cutting out the sugar chain using deglycosylation enzymes, and performing buffer replacement and the like, not only is a great deal of time and labor required, but the problem arises that valuable test samples end up being lost.

Furthermore, in the above-described conventional method, because the proteins being analyzed are broken down, even if it were possible to analyze information about the structure imparted by the post-translational modification such as the proteins contained in the sample and the sugar chains contained in those proteins, it is still not possible to separate and purify the protein-modified body that contains the target sugar chain and the like, so that the problem arises that it is not possible to directly analyze and effectively utilize the functions inherent in the proteins that have been produced by the steric structure and the characteristic portions.

Because of this, in spite of the fact that medicinal effects such as the immunological effects, etc., of immunoglobins and the like that are used as protein pharmaceuticals vary due to differences in the sugar chains that are bonded to the proteins, currently no separation or purification targeted at the variations and characteristics of a sugar chain structure is being performed, and there is an enormous potential for danger such as from unforeseen side effects and the like, so that a method for separating or purifying proteins having a single sugar chain structure is still being sought.

### DOCUMENTS OF THE PRIOR ART

### Patent Documents

[Patent document 1] Japanese Unexamined Patent Application (JP-A) No. 2007-010495

### Non-Patent Documents

[Non-patent document 1] “Development of a C₆₀-fullerene bonded open-tubular capillary using a photo/thermal active agent for liquid chromatographic separations by π-π interactions”, T. Kubo et al., Journal of chromatography A, 1323, 174-178 (2014)
[Non-patent document 2] “Polymer-based Photocoupling agent for the Efficient Immobilization of Nanomaterials and Small Molecules”, T. Kubo et al., Langmuir., 27 (15), 9372-9378, Aug. 2, 2011.

### DISCLOSURE OF THE INVENTION

### Problems to be Solved by the Invention

The present invention was conceived in view of the above-described problems, and it is a principal object thereof to provide a separating agent for liquid chromatography that is capable of separating biopolymers such as proteins and peptides using target characteristics as an index, while maintaining the original steric structure thereof.

### Means for Solving the Problem

A separating agent for liquid chromatography according to the present invention is equipped with a substrate, a recognition site (also known as an adsorption site) including a compound that operates by recognizing characteristics of biopolymers such as proteins, and a spacer that bonds the recognition site to the substrate, wherein the spacer has an effective length to enable the recognition site to operate by reaching deep portions of the steric structure of a target biopolymer.

According to this separating agent for liquid chromatography, because the spacer enables the recognition site to enter deep portions of the steric structure of the biopolymer, and the recognition site is thus able to recognize and adsorb characteristics of not only portions at the surface of that biopolymer, but also of portions buried deep in the steric structure, separation is possible using the target characteristics as an index without breaking down the biopolymer and while maintaining the original steric structure thereof.

An example of a specific embodiment of the present invention is one in which the length of the spacer is not less than 1 nm and not more than 50 nm.

If the spacer contains a synthetic polymer that is a polymer equal to or greater than a dimer, then controlling the length of the spacer is simple, and because the length of each spacer can be made uniform, reproducibility when performing biopolymer separation can be improved.

If the spacer contains a highly hydrophilic, synthetic polymer that contains a hydrophilic bond portion such as an ether bond, an ester bond, an amide bond, a urea bond, or a urethane bond or the like, then the spacer has excellent affinity with the solvent used to dissolve the biopolymer, and additionally, because the spacer has comparatively high flexibility, it is even easier for the recognition site to enter deep portions of the steric structure of the biopolymer.

If the recognition site is, for example, an organic π electron system compound such as a carbon microstructure body, then because the organic π electron system compound recognizes and adsorbs functional groups such as sugar chains and the like, separation of the biopolymer that contains a glycoprotein using the sugar chain as an index is possible.

If the recognition site is fullerene, then because the diameter of fullerene, which is a spherical structural body, is approximately 1 nm, and is small enough compared, for example, with a biopolymer such as a protein that the recognition site is able to easily enter deep portions of a protein having a steric structure, even if characteristic portions such as the sugar chain are buried deep in the steric structure of the protein, the sugar chain can still be recognized and adsorbed.

The fullerene referred to here is not limited to C₆₀, and may also indicate analogs such as C₇₀ and C₈₀ as well as fullerene derivatives.

An example of a specific embodiment of the present invention is one in which the substrate contains at least one or a plurality of compounds selected from a group containing metal oxides, carbon, polysaccharides, and synthetic polymers.

If the substrate contains a silica gel, then a combination of high resolution provided by the silica gel, and a resolution that uses the target characteristics as an index as provided by the recognition site enables characteristic separation to be performed with a high degree of accuracy.

If the substrate is a continuous porous silica gel, then compared with when silica gel particles are used for the substrate, because the substrate has a high level of porosity, and the high polymer holding time can be moderately shortened, separation can be performed at a high resolution further into the high polymer regions.

Furthermore, compared with when silica gel particles are used for the substrate, because the substrate has a high level of porosity, and it is possible to suppress any rise in the internal pressure of a column even if the flow rate of liquid flowing through the column is raised, biopolymer separation can be performed at even higher speeds.

The same type of effects as those obtained from the present invention can also be demonstrated using a separation column for liquid chromatography that has been filled with the separating agent for liquid chromatography according to the present invention.

The same type of effects as those obtained from the present invention can also be demonstrated using a method for separating a biopolymer that employs a separating agent for liquid chromatography that is equipped with a substrate, a recognition site that recognizes and adsorbs characteristics of biopolymers, and a spacer that binds the recognition site to the substrate, and that is characterized in that the spacer has an effective length to enable the spacer to reach deep portions of the steric structure of a target biopolymer.

### Effects of the Invention

According to the separating agent for liquid chromatography and to the separation column of the present invention, because the spacers enable compounds contained in the recognition site to enter deep portions of the steric structure of biopolymers such as proteins and peptides, and the recognition site is thus able to recognize and either adsorb or separate characteristics of not only superficial portions of that biopolymer, but also of portions buried deep in the steric structure, separation and purification of the biopolymer is possible using the target characteristics as an index without breaking down the biopolymer and while maintaining the original steric structure thereof.

### BRIEF DESCRIPTION OF THE DRAWINGS

FIG. 1 is a schematic view of protein recognition using a separating agent for liquid chromatography according to an embodiment of the present invention.

FIG. 2 is a view showing a procedure to manufacture the separating agent for liquid chromatography according to the present embodiment.

FIG. 3 is a view showing an interaction image between fullerene and sugar chains according to the present embodiment.

FIG. 4 is a view showing results of liquid chromatography according to Example 1.

FIG. 5 is a view showing results of liquid chromatography according to Example 2.

FIG. 6 is a view showing results of liquid chromatography according to Example 3.

FIG. 7 is a view showing results of liquid chromatography according to Example 4.

FIG. 8 is a view showing results of mass spectrometry according to Example 4.

FIG. 9 is a view showing results of mass spectrometry according to Example 4.

FIG. 10 is a view showing results of liquid chromatography according to Example 5.

### BEST EMBODIMENTS FOR IMPLEMENTING THE INVENTION

Hereinafter, an embodiment of the present invention will be described using the drawings.

A separation column for liquid chromatography according to the present invention is, for example, a column for liquid chromatography that is used by being mounted on a high-performance liquid chromatography (HPLC) apparatus.

The separation column for liquid chromatography is, for example, a capillary column in which a separating agent 1 for liquid chromatography is held inside a commercially available glass capillary tube having an internal diameter of 0.1 mm, and a length of approximately 30 cm.

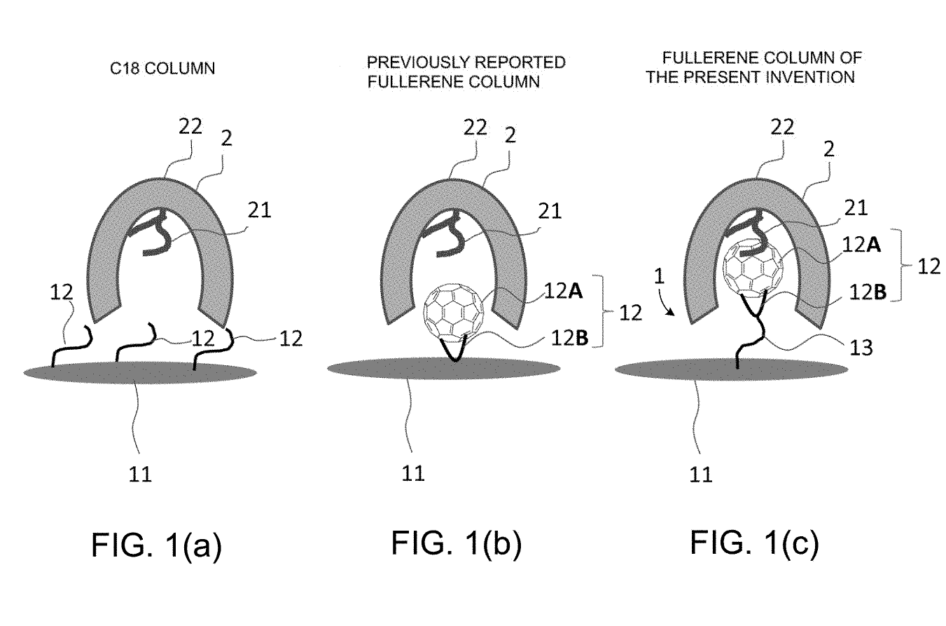

As is shown in FIG. 1(c), the separating agent 1 for liquid chromatography is provided with a substrate 11 that forms a framework for the separating agent 1 for liquid chromatography, a recognition site (also called an adsorption site) 12 that recognizes and adsorbs, for example, a sugar chain 21, which is a characteristic portion of, for example, a glycoprotein 2, and a spacer 13 that bonds the substrate 11 to the recognition site 12.

The substrate 11 has, for example, silica gel as the principal constituent thereof and, in the present embodiment, monolithic silica gel, which is a continuous porous body having a three-dimensional network structure is used. In the present embodiment, a substrate formed by bonding, for example, (3-triethoxysilyl) propylsuccinic anhydride (may also be referred to below as silane), which is a silane coupling agent, to the surface of the silica gel is used as the substrate 11.

The recognition site 12 is fixed to the surface of the substrate 11, for example, to the interior of fine pores of the monolithic silica gel in order to impart characteristic adsorption properties to the separating agent 1 for liquid chromatography, and is provided with a compound 12A that operates by recognizing characteristics of a biopolymer, and with a binding reagent 12B that fixes this compound to the spacer 13.

In this embodiment, the compound 12A that operates by recognizing the characteristics of a biopolymer is, for example, fullerene C₆₀, which is a type of carbon microstructural body.

The fullerene C₆₀ is spherical graphite carbon having a diameter of approximately 1 nm, and it is thought that it is able, for example, to recognize and adsorb the sugar chain 21 present in the glycoprotein 2 because π electrons are abundant on the surface thereof.

The amount of immobilized fullerene is set, for example, such that 15 parts by weight of fullerene is immobilized relative to 100 parts by weight of silica gel, which is serving as the substrate 11.

In this embodiment, the binding reagent 12B is, for example, 4-azido 2,3,5,6-tetrafluorobenzoic acid (may also be referred to below as PFPA).

The spacer 13 enables, for example, the fullerene contained in the recognition site 12 to reach the sugar chain 21 buried in deep portions of the steric structure of a target biopolymer, for example, the glycoprotein 2. The spacer 13 has a length of, for example, approximately 2 nm, and one end thereof is bound to the substrate 11, while another end thereof is bound to the recognition site 12.

A suitable material for the spacer 13 is a straight-chain hydrophilic synthetic polymer that has flexibility such as, for example, polyethylene glycol (may also be referred to below as PEG).

Furthermore, a bond group that is used respectively to bond to the substrate 11 and the recognition site 12 is provided at both ends of this spacer 13.

Examples of this bond group include a hydroxyl group, an amino group, a carboxyl group, and the like.

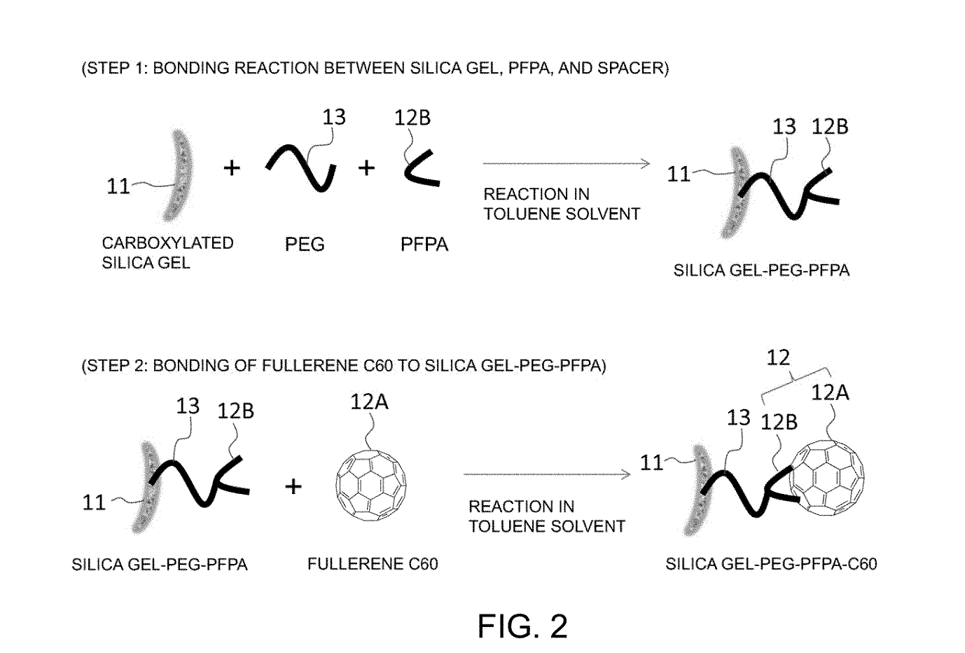

The separating agent 1 for liquid chromatography can be manufactured via a process such as that shown in FIG. 2.

Specifically, firstly, a capillary column holding the monolithic silica gel serving as the substrate 11 was washed with 1.0 M HCl and water. After the silanol group of the substrate 11 was activated, the silanol group was replaced with methanol, and the interior of the capillary was dried with nitrogen gas. At this point, silane diluted with a 10% toluene solvent (v/v) was supplied thereto, so that the silane was bonded to the surface of the monolithic silica gel.

Subsequently, a reagent for example, a toluene solution (10% v/v) obtained by binding, for example, the PFPA serving as the binding reagent 12 to, for example, the PEG serving as the spacer 13 was supplied for 24 hours at a flow rate of 10 μl/h.

Next, for example, 36 mM of a fullerene C₆₀ solution serving as the compound 12A that operates by recognizing the characteristics of the biopolymer in which, for example, toluene was used as the solvent was used to fill the interior of the capillary, and this was then left at room temperature for 24 hours. It was subsequently heated for 72 hours at 140° C.

Finally, a solvent such as dichlorobenzene was supplied thereto, and any fullerene C₆₀ that did not react was removed.

The sequence in which the compound 12A that operates by recognizing the characteristics of the biopolymer, the binding reagent 12B, the spacer 13, and the substrate 11 are bonded together is not limited to that of the above-described procedure, and they may be bonded together in any sequence.

The separation method for a biopolymer that employs liquid chromatography using the separating column 1 for liquid chromatography created in this manner is described below.

The capillary column mounted in a gradient-type nanoflow liquid chromatography apparatus (an Ultimate 3000 Nano, Dionex Corporation) is firstly washed with the eluent to be used for the analysis, and a test sample containing the glycoprotein 2, which is the target biopolymer, is introduced into the capillary column.

The biopolymer which is serving as the target in this case is a biopolymer such as the glycoprotein 2 that is to be subjected to separation, and undergoes separation as a result of being recognized and adsorbed by the recognition site 12.

Next, ultra-pure water containing 0.1% by volume ratio of TFA, as well as an eluent obtained from 1-propanol are supplied to the capillary column at a flow rate of, for example, 300 nl/min, and the glycoprotein 2 eluted by the eluent is detected using a fluorescence detector, an ultraviolet absorbance detector, or a mass spectrometer that is provided in the liquid chromatography apparatus.

At this time, the task of cleaning the capillary column, the introduction of the test sample, the introduction of the eluent, and the control of the flow rates and the like are performed using a control device such as a pump provided in the liquid chromatography apparatus, and a control program.

According to the separation column 1 for liquid chromatography having the above-described structure, because the fullerene spatially disposed on the surface of the silica gel or inside the micropores thereof by the spacer 13 recognizes and adsorbs not only the sugar chains 21 that are on the surface of the target glycoprotein 2, but also the sugar chains 21 that are present in deep portions of the steric structure thereof, it is possible to perform separation using liquid chromatography without breaking down the glycoprotein 2, and while maintaining the original steric structure thereof.

Because polyethylene glycol is used for the spacer 13, it is possible to raise the affinity of the substrate 11 towards an aqueous solvent without damaging the π electrons in the fullerene serving as the recognition site 12.

Because the length of the spacer 13 is approximately the length of C16, it is easy for the fullerene serving as the recognition site 12 to enter the deep portions of the steric structure of the comparatively large glycoprotein 2.

Because the spacer 13 is made from polyethylene glycol, it has a high degree of structural flexibility, and the fullerene can easily enter the deep portions of the steric structure of the glycoprotein 2.

Because the spacer 13 is a polymer of polyethylene glycol, it is easy to control the length of the spacer 13, and easy to control the length of the spacer 13 such that this length is a target length, and also to uniformize the length of each spacer 13.

Furthermore, because polyethylene glycol is a substance that has a low level of biological toxicity, it is easy to handle.

Furthermore, the diameter of the fullerene serving as the recognition site 12 is approximately 1 nm and is small enough compared to the size of the target glycoprotein 2 that it is easily able to enter deep portions of the steric structure of the glycoprotein 2.

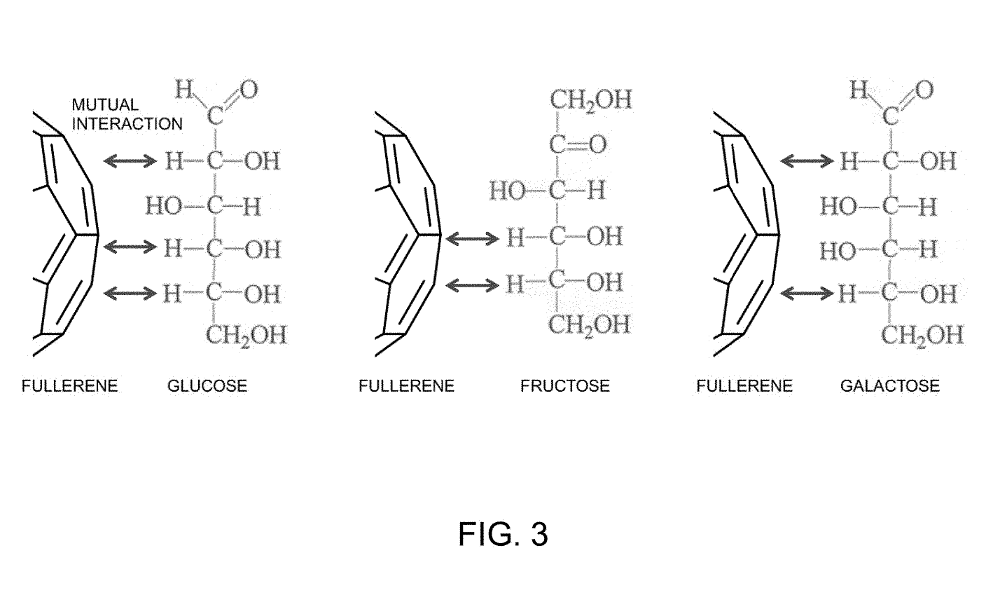

As is shown in FIG. 3, it is thought that fullerene is able, for example, to recognize CH groups in the sugar chains 21 via the π electrons that are abundant on the surface thereof, and because the adsorption efficiency thereof is changed by the number and direction and the like of the CH groups contained in the sugar chains 21, and this enables differences between sugar chains 21 to be identified, the glycoprotein 2 can be accurately separated using the type of sugar chain 21 and the number thereof.

Because the glycoproteins 2 can be accurately separated using differences between the sugar chains 21, the quality of the glycoprotein 2 as a biopharmaceutical can be improved.

This will now be described in more detail. Glycoproteins 2 are eagerly anticipated as, for example, an antibody drug, an immunosuppressive agent, and an anti-cancer agent and the like, and the medical efficacy thereof depends on a protein portion 22. It is thought that the effectiveness of the glycoprotein 2 is determined by the structure and the like of the sugar chain 21 portion thereof.

However, recognizing differences between these sugar chain 21 portions in the glycoprotein 2 and accurately separating them is difficult, and sugar chains 21 having different structures and numbers are mixed together in the glycoproteins 2 conventionally used as biopharmaceuticals.

According to the separating agent 1 for liquid chromatography according to the present invention, because it is possible to accurately perform separations using differences between the structures and numbers of the sugar chains 21, improving their quality as biopharmaceuticals is possible, and it is also thought that even tighter control of their medical efficacy and the like will be possible.

Because chemically modifying the fullerene enables differing adsorption characteristics to be imparted thereto, changing or adjusting the adsorption capabilities thereof is also possible.

Because the separating agent 1 for liquid chromatography according to the present embodiment can be used by being held in a commercially available capillary column that is capable of being used in a high-performance liquid chromatography apparatus, the glycoprotein 2 can be separated while maintaining the original steric structure thereof via a simple operation performed using a conventional liquid chromatography apparatus.

Moreover, the liquid chromatography apparatus can be connected, for example, online to a high-resolution mass spectrometer.

Because monolithic silica gel, which is a continuous porous silica gel body, is used as the substrate 11, it is possible to suppress any rise in the pressure inside a capillary column compared to when a granular silica gel is used for the substrate 11, so that the separation of the glycoprotein 2 can be performed at a higher speed.

Note that the present invention is not limited to the above-described embodiment.

For example, in the above-described embodiment, the glycoprotein 2 was the target biopolymer; however, by changing the recognition site 12, proteins and the like having a variety of characteristics may form the target biopolymer.

The substrate 11 is not limited to being a monolithic silica gel, and all-porous silica gel particles, superficial porous silica gel particles, or non-porous silica gel particles may also be used. Moreover, the substrate 11 is not limited to being a silica gel, and metal oxides such as glass, titania, zirconia, and alumina and the like, polysaccharides such as agarose, dextran and derivatives of these, polymers such as acrylamide, poly(styrene/divinylbenzene) and the like, as well as compounds having carbon materials such as activated carbon and the like as a principal constituent thereof, and various different combinations of these may also be used.

A capillary column is employed for the configuration of the column for liquid chromatography; however, the present invention is not limited to employing a capillary column, and it is also possible, for example, to employ a rod-type column that is capable of being mounted in a liquid chromatography apparatus.

Moreover, the present invention is not limited to employing a packed column, and it is also possible for a user to fill an open column or a spin column with the separating agent 1 for liquid chromatography according to the present invention when using the present invention.

Fullerene C₆₀ is used for the recognition site 12; however, other types of fullerene may also be used. Furthermore, the present invention is not limited to using fullerene, and carbon microstructures such as carbon nanotubes and graphene and the like that, in the same way as fullerene, have π electrons, and have the capacity to recognize and adsorb the sugar chains 21 may also be used.

Because the properties of fullerene can be altered by subjecting the fullerene to chemical modification, it is possible, for example, to achieve an improvement in the ability thereof to adsorb the sugar chains 21, and an improvement in the recognition specificity thereof and the like.

The amount of immobilized fullerene is not limited to being 15 parts by weight relative to 100 parts by weight of the substrate 11, and provided that the amount is not less than 5 parts by weight and not more than 50 parts by weight of fullerene relative to 100 parts by weight of the substrate 11, then the sugar chains 21 can be adequately recognized and adsorbed.

Because it is possible to change the ability thereof to adsorb the sugar chains 21 by increasing or reducing the amount of immobilized fullerene, the separation characteristics of the separating agent 1 for liquid chromatography can be adjusted.

Additionally, the recognition site 12 is not limited to these carbon microbodies, and natural or synthetic compounds having a high level of affinity towards the biopolymer or the sugar chains 21 such as lectin may also be used.

The spacer 13 is not limited to being polyethylene glycol, and any hydrophilic polymer that contains a hydrophilic bond portion such as an ether bond, an ester bond, an amide bond, a urea bond, or a urethane bond may be used.

The bond group is also not limited to being a hydroxyl group, an amino group, or a carboxyl group, and it is sufficient if one end of a straight-chain hydrocarbon portion can be bonded to the substrate 11, and the other end thereof can be bonded to the recognition site 12.

The length of the spacer 13 is not limited to 2 nm, and it is sufficient if this length is not less than 1 nm and not more than 50 nm.

Furthermore, if the length of the spacer 13 is not less than 1 nm and not more than 10 nm, then this is preferable as the adsorption agent is able to more easily reach deep portions of the steric structure of a target biopolymer.

For example, if polyethylene glycol is used for the spacer 13, then the length of the polyethylene glycol dimer is approximately 1 nm.

In addition to these, the present invention is not limited to the above-described embodiment, and various modifications thereof are possible insofar as they do not depart from the spirit or scope thereof.

Hereinafter, the present invention will be described in further detail while providing examples; however, the present invention is not limited solely to these examples.

### Example 1

In Example 1, the separation characteristics of proteins were examined using a silica monolithic capillary column in which fullerene has been immobilized via a spacer.

In the column used in the present example, the separating agent 1 for liquid chromatography onto which fullerene C₆₀ has been fixed is supported via PFP-A-PEG-silane on monolithic silica gel which is serving as the substrate 11.

In the present example, a column in which the spacer 13 was PEG200 (having a length of approximately 2 nm) was used. Hereinafter, this column is referred to as a PEG200-C60 bonded column.

In addition, a column in which fullerene C₆₀ was fixed directly onto the monolithic silica gel without a spacer being interposed (hereinafter, this may also be referred to as a C60 bonded column), and a C18 column were used as comparative columns.

The size of the columns in the present example was set at an inner diameter of 0.1 mm and a length of 30 cm.

In this example, a 0.11 mg/mL bovine serum albumin aqueous solution (Sigma-Aldrich Japan) was used as the test sample.

Bovine serum albumin is a protein without a sugar chain that is widely used as a model protein in the field of protein research and has a molecular weight of approximately 67,000.

Additionally, it is also known that bovine serum albumin is a protein having a comparatively high level of hydrophobicity.

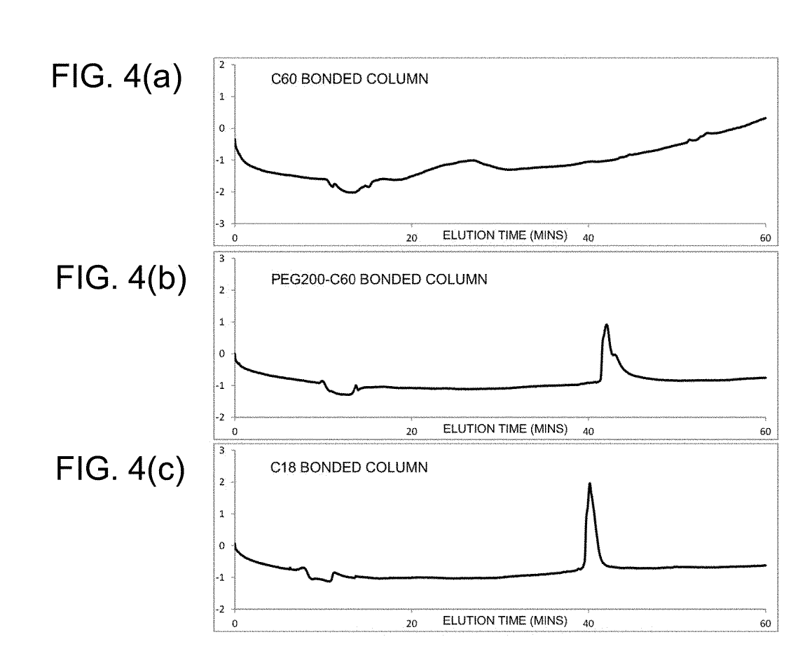

The results obtained when liquid chromatographic separation was performed on this bovine serum albumin in a PEG200-C60 bonded column, a C60 bonded column, and a C18 bonded column are shown respectively in FIGS. 4(a), (b), and (c).

The horizontal axis shows elution time, while the vertical axis shows the spectral absorbance of the eluted protein.

A gradient-type nanoflow liquid chromatography apparatus (an Ultimate 3000 Nano, Dionex Corporation) was used as the high-performance liquid chromatography apparatus.

Additionally, the following conditions were applied as the analysis conditions:

Sample injection amount: 0.5 μl

Mobile phase (A): water+formic acid (1% v/v)

Mobile phase (B): (acetonitrile/isopropanol=50/50)+formic acid (1% v/v)

Supply flow rate: 300 nl/min

Column temperature: 60° C.

Detection: UV 280 nm

Gradient conditions: 3%-43% B (60 mins)

As is shown in FIG. 4(a), in the C60 bonded column in which fullerene C₆₀ was bonded directly onto the monolithic silica gel without a spacer being interposed therebetween, a bovine serum albumin peak could not be detected.

In contrast, as is shown in FIG. 4(b), in the PEG200-C60 bonded column in which fullerene C₆₀ was bonded onto the monolithic silica gel via a spacer, a peak that was thought to be bovine serum albumin was detected.

As is shown in FIG. 4(c), the results from the PEG200-C60 bonded column strongly matched the elution pattern of the bovine serum albumin in the C18 column that is generally used in separation and purification.

It is thought that the main reasons why bovine serum albumin that could not be eluted in the C60 bonded column was able to be extracted without any problem in the PEG200-C60 bonded column were that, for example, in the PEG200-C60 bonded column in which a hydrophilic spacer was used, compared with the C60 bonded column in which no spacer was used, due to the effects of the hydrophilic spacer, the bond with the protein did not become too strong, and also that it was easy for the eluent to intrude into the spacer portion, and the like.

From the results described above, according to the column in which fullerene C₆₀ was immobilized via a spacer on monolithic silica gel, it was found that proteins could be separated at an equivalent separation performance as that obtained from a C18 column which is generally used in conventional protein separation and purification.

Moreover, according to the column in which fullerene C₆₀ was immobilized via a spacer on monolithic silica gel, it was also found that, even when bovine serum albumin which is highly hydrophobic and whose degree of attachment to the column is strong was used, separation was still possible under comparatively mild conditions.

### Example 2

Next, in Example 2, the separation characteristics of glycoproteins were examined using the above-described silica monolithic capillary column containing immobilized fullerene.

The column used in this example was a PEG200-C60 column having an inner diameter of 0.1 mm and a length of 31 cm.

A C18 column having an internal diameter of 1 mm and a length of 31 cm was used as a comparative column.

Conalbumin (Sigma-Aldrich Japan) having an antiviral action and an antibacterial action was used as the test sample.

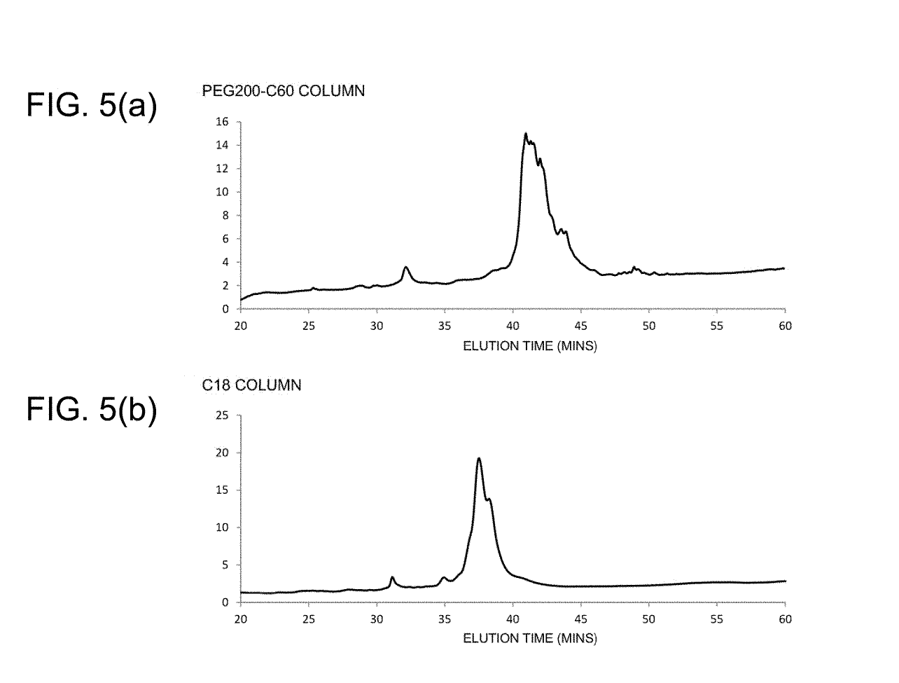

Conalbumin (also known as ovotransferrin) is a glycoprotein having a molecular weight of approximately 76,000 and contains approximately 2% of its weight in the form of sugar chains. The results obtained when liquid chromatographic separation was performed on this conalbumin in a PEG200-C60 bonded column, and a C18 column are shown respectively in FIGS. 5(a) and (b).

The horizontal axis shows elution time, while the vertical axis shows the spectral absorbance of the eluted glycoprotein.

A gradient-type nanoflow liquid chromatography apparatus (an Ultimate 3000 Nano, Dionex Corporation) was used as the high-performance liquid chromatography apparatus.

Additionally, the following conditions were applied as the analysis conditions:

Mobile phase (A): H2O/TFA=100/0.1

Mobile phase (B): 1-propanol/H2O/TFA=90/10/0.1)

Supply flow rate: 300 nl/min

Column temperature: 60° C.

Detection: UV 214 nm

Gradient conditions:
(fullerene column) 3% B-(2 mins)-3% B-(3 mins)-17% B-(50 mins)-30% B
(C18 column) 3% B-(2 mins)-3% B-(3 mins)-23% B-(50 mins)-36% B

From the results shown in FIG. 5 it was found that, relative to the C18 column that is generally used in separation and purification, in a fullerene column in which PEG200 is used as a spacer, irrespective of the fact that the same sample was used, the number and behavior of detection peaks was different compared to the C18 column.

From these results it was confirmed that, if the separation column for liquid chromatography according to the present invention is employed, structural changes in deep portions of a glycoprotein where action has not conventionally been possible were recognized, and separation of the glycoproteins in an unmodified form was possible without the glycoproteins being broken down.

### Example 3

Next, the separation characteristics of antibody proteins were examined by performing liquid chromatography using the separation column for liquid chromatography according to the present invention.

Bovine serum γ-globulin (Nacalai Tesque) used in the model research of antibody drugs was used as the test sample.

γ-globulins are an antibody protein having a molecular weight of approximately 150,000 and are known as an immunoglobulin. Because the immune effect of γ-globulins varies due to differences in the structures thereof caused by post-translational modifications such as sugar chains, γ-globulins produced using a specific manufacturing method are used as antibody drugs.

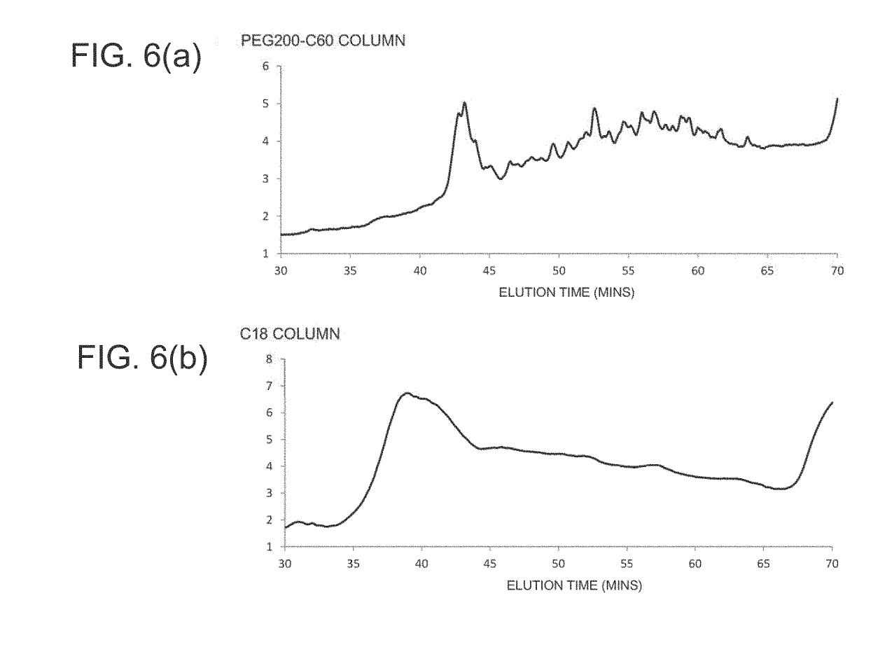

The results obtained when liquid chromatographic separation was performed on these γ-globulins in a PEG200-C60 bonded column, and a C18 column are shown respectively in FIGS. 6 (a) and (b).

The horizontal axis shows elution time, while the vertical axis shows the spectral absorbance of the eluted protein.

The analyzing apparatus and the analysis conditions were the same as those employed for Example 2.

From the results shown in FIG. 6 it was found that, compared to the C18 column, in a column in which fullerene was bonded using a PEG spacer, the number and elution behavior of detection peaks was different.

From this it was confirmed that, if the separation column for liquid chromatography according to the present invention is employed, structural changes in deep portions of an immunoglobulin where action has not conventionally been possible were recognized, and separation of the immunoglobulins was possible.

### Example 4

In Example 4, liquid chromatography was performed using a PEG600-C60 bonded column in which PEG600 was used for the spacer, and mass spectrometry was further performed on the eluted proteins to confirm whether separation of the glycoproteins had been achieved.

A monoclonal antibody, mAb check standard (0.1 mg/mL, Waters Inc.), some of which are known to have various sugar chain structures was used as the test specimen.

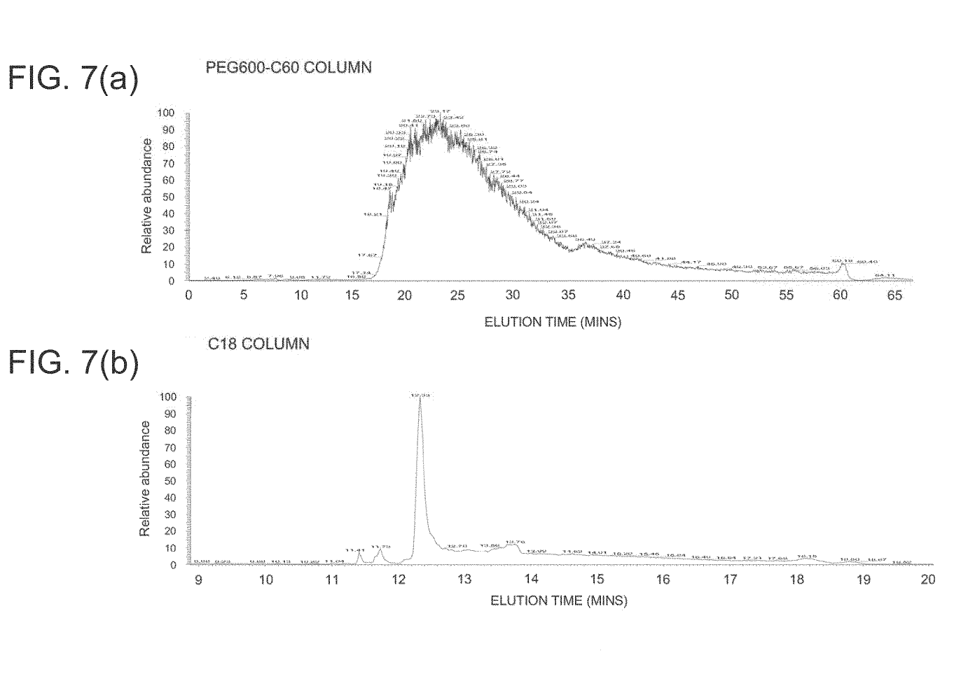

The results obtained when liquid chromatographic separation was performed on these monoclonal antibodies in a PEG600-C60 bonded column, and a C18 column are shown respectively in FIGS. 7 (a) and (b).

Note that in FIG. 7, the horizontal axis shows elution time, while the vertical axis shows the ion detection values of the eluted protein.

The size of the columns in the present example was set at an inner diameter of 0.1 mm and a length of 250 mm.

The analyzing apparatus was the same as that used in Example 1.

Additionally, the following conditions were applied as the analysis conditions:

Sample injection amount: 0.5 μl

Mobile phase (A): water+1% formic acid (v/v)

Mobile phase (B): (acetonitrile/isopropanol=50/50)+1% formic acid (v/v)

Supply flow rate: 400 nl/min

Column temperature: 60° C.

Detection: UV 280 nm

Gradient conditions: 25%-35% B (50 mins)

As is shown in FIG. 7, from the results of this liquid chromatography a broad peak was confirmed in the PEG600-C60 column, while a sharp peak together with tailing were confirmed in the C18 column.

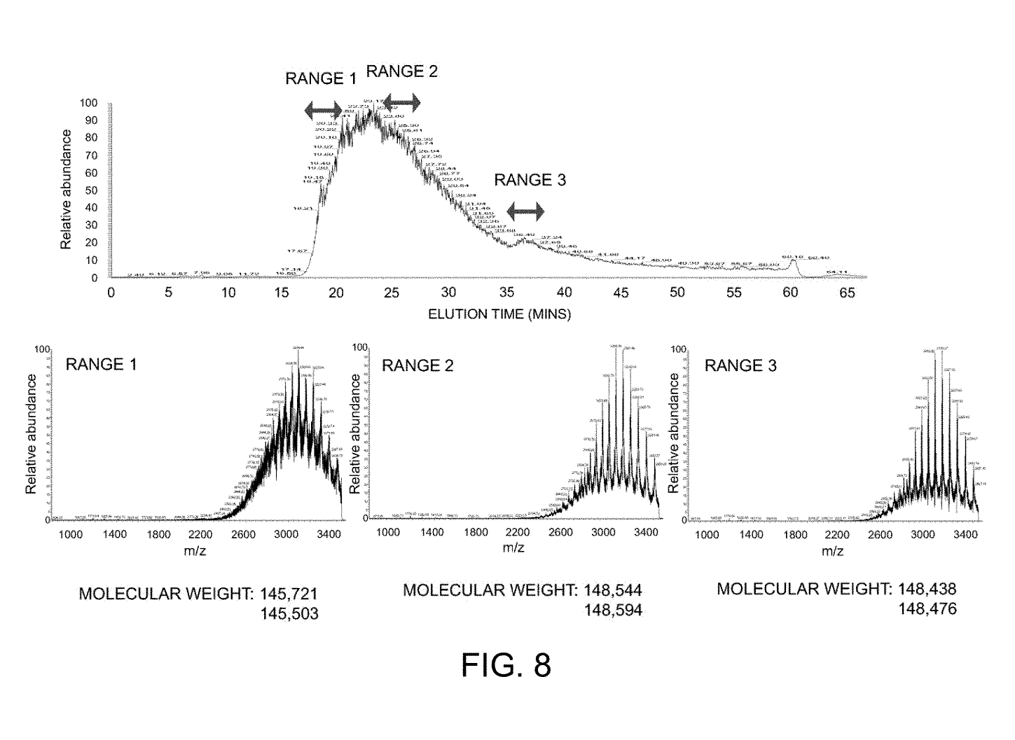

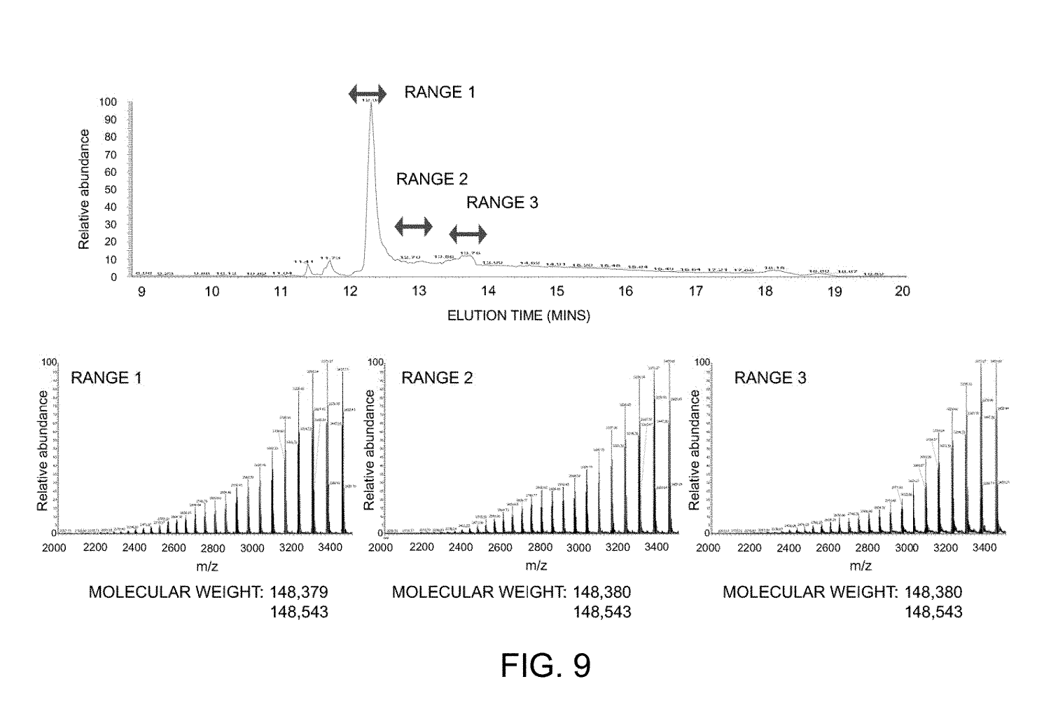

Next, as is shown in FIG. 8 and FIG. 9, the mass-to-charge ratios (m/z) of the respective protein fractions in each of range 1, range 2, and range 3 was measured for each one of FIGS. 7(a) and (b) using a Fourier transform high-resolution mass spectrometer (Exactive Plus Orbitrap, Thermo Fisher Scientific Inc.)

The analysis conditions for the mass analysis were as follows.

Scan range: 800-3,000 m/z

Fragmentation: Insource CID 60.0 eV

Resolution: 17,500

Polarity: Positive

Microscans: 10

AGC: 5e6

Spray voltage: 2.15 kV

Capillary temperature: 325° C.

S-Lens: 90.0

Based on the obtained mass-to-charge ratios (m/z), the molecular weights of the proteins were determined by performing deconvolution processing using software (Thermo Biopharma Finder 1.0).

The conditions for this deconvolution processing were as follows.

Intact Protein Analysis Mode

m/z range: 2,000-3,000

Output mass range: 100,000-160,000

Mass tolerance: 20 ppm

Target mass: 150,000

Charge range: 10-100

As is shown in FIG. 8, when the peaks obtained in the PEG600-C60 column were extracted in range 1˜range 3 and mass spectrometry was performed, different mass information was obtained from the ranges.

From the mAb Check Standard test sample catalog information, the protein molecular weight when the protein contains no sugar chains is approximately 145,000, and this molecular weight becomes approximately 148,000-149,000 as a result of bonding with a sugar chain.

The molecular weight obtained in range 1, which is in front of the peak in FIG. 8, is 145,000, and it is thought that proteins dissociated from a sugar chain are being eluted. In contrast, the molecular weight in ranges 2 and 3, which are in the rear half of the peak, is approximately 148,000, and because different molecular weight values are shown in range 2 and range 3, it is thought that monoclonal antibodies having different sugar chain structures are being separated.

In contrast to this, as is shown in FIG. 9, in the peak ranges 1˜3 obtained from the C18 column, substantially the same molecular weight values were shown in range 1, which is the main peak, as in range 2 and range 3, which continued on after range 1.

Because of this, it is thought that the changes in the shape of the peak and the elution behavior seen in the C18 column were due to the fact that no separation took place because of differences in the molecular weight (i.e., differences between the types of sugar chain and the like).

From the above results it was shown that a fullerene-bonded column using a hydrophilic spacer had the characteristic of separating and purifying glycoproteins present in an aqueous solvent based on the type and structure of the sugar chains.

### Example 5

Next, an experiment to recognize differences in the sugar chain was performed via liquid chromatography using the separation column for liquid chromatography according to the present invention.

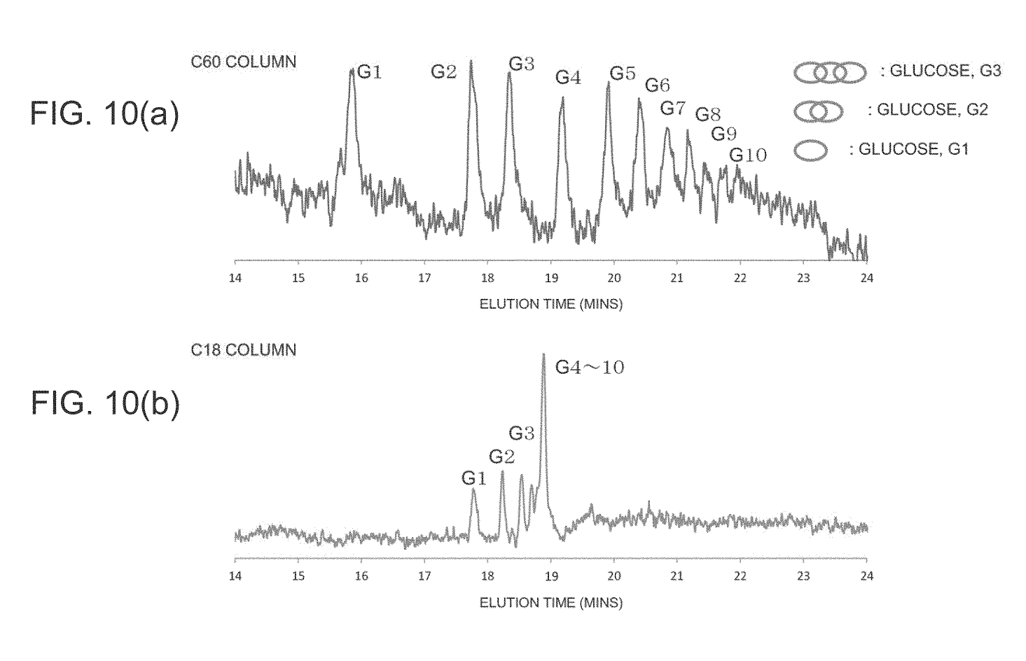

Separations were attempted using a fullerene-bonded capillary column having no spacer (hereinafter, this may also be referred to as a C60 bonded column) as well as a C18 column, and using sugar chains having various lengths from a glucose monosaccharide to an oligosaccharide having 23 continuous glucose units as test samples, and under the separation conditions used for the glycoprotein analysis. The results when these separations were attempted are shown in FIGS. 10(a) and (b).

Here, the numbers included in the sample names represent the number of glucose bonds. For example, G1 represents a glucose monomer, and G2 represents a glucose dimer.

Furthermore, the chromatography conditions were as follows, namely, equivalent conditions as those employed for the glycoprotein analysis were applied.

Note that a gradient-type nanoflow liquid chromatography apparatus (an Ultimate 3000 Nano, Dionex Corporation) was used as the liquid chromatography apparatus.

Mobile phase (A): H₂O/TFA=100/0.1

Mobile phase (B): acetonitrile/H₂O/TFA=90/10/0.1)

Flow rate: 500 nl/min

Column temperature: 40° C.

Detection wavelength: 330 nm

Sample: 2-AB glucose homopolymer

Injection amount: 500 nl

Gradient: 5-50% B (80 mins linear)

As is shown in FIG. 10(a), in the fullerene-bonded capillary column according to the present invention, it was found that, in an oligosaccharide in which multiple glucose units were connected together as more or less a glucose decamer, the length of the sugar chain could be recognized with a high level of sensitivity and separated. In contrast to this, as is shown in FIG. 10(b), in the C18 capillary column, sharp peaks were verified for the monosaccharide and disaccharide oligosaccharides, and although separation of these was possible, separation was not possible in the sugar chains having higher molecular weight than these.

In this way, according to the fullerene-bonded type of separation column for liquid chromatography according to the present invention, it was found that, in the protein separation conditions, differences in the sugar chains could be recognized with an extremely high level of sensitivity and separated.

### LIST OF REFERENCE CHARACTERS

1 . . . Separating agent
11 . . . Substrate
12 . . . Recognition site
12A . . . Compound that operates by recognizing characteristics of a biopolymer
13 . . . Spacer
2 . . . Protein

1. A separating agent for liquid chromatography comprising:
a substrate;
a recognition site including a compound that operates by recognizing characteristics of biopolymers; and
a spacer that bonds the recognition site to the substrate,
wherein the spacer has an effective length to enable the recognition site to operate by reaching deep portions of the steric structure of a target biopolymer.

2. The separating agent for liquid chromatography according to claim 1, wherein the length of the spacer is not less than 1 nm and not more than 50 nm.

3. The separating agent for liquid chromatography according to claim 1, wherein the spacer includes a polymer equal to or greater than a dimer.

4. The separating agent for liquid chromatography according to claim 1, wherein the polymer serving as the spacer contains an ether bond, an ester bond, an amide bond, a urea bond, or a urethane bond.

5. The separating agent for liquid chromatography according to claim 1, wherein the recognition site contains an organic π electron system compound.

6. The separating agent for liquid chromatography according to claim 1, wherein the recognition site includes fullerene.

7. The separating agent for liquid chromatography according to claim 1, wherein the substrate contains at least one or a plurality of compounds selected from a group containing metal oxides, carbon, polysaccharides, and synthetic polymers.

8. The separating agent for liquid chromatography according to claim 1, wherein the substrate contains a silica gel.

9. The separating agent for liquid chromatography according to claim 1, wherein the substrate contains a continuous porous silica gel.

10. A separation column for liquid chromatography that has been filled with the separating agent for liquid chromatography according to claim 1.

11. A method for separating a biopolymer that employs a separating agent for liquid chromatography that comprises a substrate, a recognition site including a compound that operates by recognizing characteristics of biopolymers, and a spacer that bonds the recognition site to the substrate, the method comprising:
providing the spacer with an effective length to enable the recognition site to operate by reaching deep portions of the steric structure of a target biopolymer.

12. The separating agent of claim 1, wherein the biopolymers are proteins.
